(19) United States
(12) Patent Application Publication
(10) Pub. No.: US 2010/0280314 A1
(43) Pub. Date: Nov. 4, 2010

(54) UROLOGICAL RESECTOSCOPE COMPRISING HOLES

(76) Inventor: Pieter Brommersma, Bargteheide (DE)

Correspondence Address: OSTROLENK FABER GERB & SOFFEN, 1180 AVENUE OF THE AMERICAS, NEW YORK, NY, 100368403, US

(21) Appl. No.: 12/677,401

(22) PCT Filed: Nov. 19, 2008
(86) PCT No.: PCT/EP2008/009766

§ 371 Date: May 21, 2010

(30) Foreign Application Priority Data
Nov. 20, 2007 (DE) 10 2007 055 582.4

(51) Int. Cl. A61B 1/00

(52) U.S. Cl. 600/105

(57) ABSTRACT

A resectoscope comprising a shaft that has an external tube and an internal tube. A lens system which has an angular field of view and can be rotated relative to the external tube along with the internal tube is disposed inside the internal tube. The distal end region of the internal tube is designed to slide in a sealed manner along the external tube. The external tube has holes in the distal end region proximal to the end region of the internal tube. The interior of the internal tube is connected as a supply duct while the intermediate space between the tubes is connected as a discharge duct. The disclosed resectoscope is characterized in that the holes are distributed along the circumference, while the internal tube is fitted with a covering device which covers the holes in a circumferential zone.

The invention relates to a urological resectoscope of the type mentioned in the preamble of claim 1.

These days, such resectoscopes are the standard instrument from all relatively large manufacturers of urological instruments. They are used for endoscopic operations in the bladder and especially for resecting the hypertrophic prostate. For this, an instrument which is usually designed as a radiofrequency-actuated cutting loop is arranged in the inner tube and the resection is performed using said instrument whilst being observed by means of the optical system with the oblique field of view.

In the process, the continuous rinsing ensured by the supply duct and the discharge duct, which is present at the same time, provides for good visibility and rinses clouding, especially blood, from the field of view. The rinsing liquid emanates from the inner space of the inner tube which forms the supply duct and is led away through the holes in the outer tube, from where the liquid can flow out through the intermediate space in the tube. The seal between the tubes provided at the distal end of the inner tube ensures that the liquid can, in a targeted fashion, only flow out through the holes.

The ability of the inner tube to rotate together with the optical system, wherein the utilized instrument can also be rotated together with these, ensures that only the inner parts are rotated during the frequent reorientations whilst working with the cutting instrument, while the outer tube can remain stationary in the urethra and so damage to the urethra as a result of constant rotating to and fro can be avoided.

However, the major advantage of the generic design, namely the ability of the outer tube to rotate with respect to the inner tube, is the cause of a disadvantage in known generic designs. In the distal end region, the shaft has a completely rotationally symmetric design. This means that the liquid flowing in via the supply duct is distributed to all sides and flows back into the holes in a rotationally symmetric fashion. Thus, the inflow is in the axial direction of the shaft. However, the viewing direction of the optical system is at an angle in order to be able to have a better overview of the work space. Since the inflowing clear rinsing liquid does not flow in the direction of the viewing direction, the visual range is limited. Clouding such as hemorrhaging is not rinsed clear over a sufficient visual range.

This is a disadvantage compared to older instruments in which the tubes are rotationally fixed with respect to one another and in which the holes are arranged asymmetrically over the circumference of the shaft in order to result in a preferred rinsing direction in the direction of the viewing direction.

The object of the present invention consists of improving the visual range in a generic resectoscope in the case of clouding.

This object is achieved by the features of the characterizing part of claim 1.

According to the invention, the holes are distributed over the circumference but are covered in a circumferential region by a covering device which rotates with the inner tube. By contrast, the holes remain open in another circumferential region. This affords the possibility of setting the flow pattern such that the rinsing direction is always in the direction of the viewing direction of the optical system, independent of the rotational position of the inner tube with respect to the outer tube.

Advantageously, provision is made of the features of claim 2 which result in a particularly simple design.

In the drawing, the invention is illustrated in an exemplary and schematic fashion. Herein,

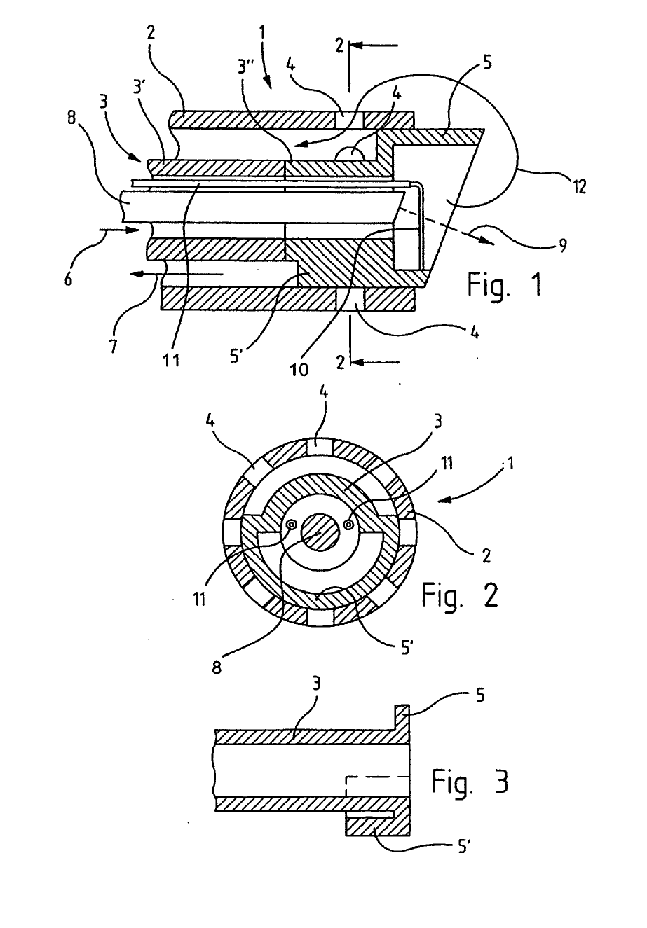

FIG. 1 shows an axial section through the distal end region of the shaft of a resectoscope according to the invention,

FIG. 2 shows a section according to the line 2-2 in FIG. 1, and

FIG. 3 shows an axial section through the end region of an inner tube in an embodiment variant with respect to FIG. 1.

FIGS. 1 and 2 show the end region of the shaft 1 of a urological resectoscope with the remaining components thereof, which can correspond to the conventional prior art, not being illustrated. The shaft 1 has an outer tube 2 and an inner tube 3, wherein the outer diameter of the inner tube 3 is smaller than the inner diameter of the outer tube 2 and so there is an intermediate space between the tubes.

The outer tube 2 has a ring of holes 4. In the exemplary embodiment, the inner tube 3 is for electrical reasons subdivided into a proximal part 3′ made of metal and a distal part 3″ made of insulating material, e.g. ceramics. Like the proximal part 3′ of the inner tube 3, the distal part 3″ likewise leaves a sufficient gap to the outer tube 2. However, the distal end region of part 3″ is designed as an end region 5 with an enlarged diameter for sliding on the inner surface of the outer tube 2 in a sealing fashion. The region 5 with the enlarged diameter starts so far distally that holes 4 in the external diameter 2 remain open.

At the proximal end of the shaft 1 (not illustrated), the inner space of the inner tube 3 is connected to a rinsing liquid supply which guides liquids through the interior of the inner tube 3 in the direction of the arrow 6. The intermediate space between the tubes 2, 3 is connected to a discharge device (not illustrated) and so liquid flows out of said space in the direction of the arrow 7. Thus, liquid flows centrally into the region in front of the distal end of the shaft 1, and said liquid flows out again through the holes 4 on the outer side of the outer tube 2.

Arranged in the interior of the inner tube 3 is an optical system 8 which is designed as an elongate tube which includes an image guiding device which can be designed as a lens arrangement, a fiber-glass bundle or a video device. The optical system 8 is designed with an oblique field of view. Thus, the viewing direction thereof, illustrated in FIG. 1 by the dashed arrow 9, is at an angle to the axis of the shaft 1. Moreover, a work instrument is arranged in the interior of the inner tube 3 and is, in the exemplary embodiment, illustrated as a conventional cutting loop 10 which is held by loop holders 11 via which radiofrequency voltage is supplied to the cutting loop 10 from a proximal connector (not illustrated).

The inner tube 3, the optical system 8 and the instrument 10, 11 are connected to one another in a rotationally fixed fashion at the proximal end of the shaft 1 (not illustrated), but together they are mounted such that they can rotate with respect to the outer shaft. This affords the possibility of the outer shaft 2 remaining stationary in the urethra in a rotationally fixed manner during work using the resectoscope, for example during the prostate resection, while the entire insert can be continuously rotated with respect to the outer tube 2 in order to be able to cut using the loop 10 at different angular positions.

If the holes 4 were freely accessible in a rotationally symmetric fashion, this would result in a central inflow along the axis of shaft 1 in the case of an inflow through the interior of the inner tube 3 and the rotationally symmetric outflow through the holes 4, i.e. this would result in a flow deviating from the viewing direction of the optical system 9. The visual range in the direction of the arrow 9 would therefore be limited.

Thus, the inner tube 3 in the distal end region thereof has a rotationally asymmetric design. In the upper circumferential region (as per FIGS. 1 and 2), the end region is designed as illustrated in the upper part of FIG. 1. The end region 5 of the inner tube 3 with an enlarged diameter is situated in a sealing fashion and distally from the holes 4 in the outer tube 2, but leaves the holes 4 open.

In the lower circumferential region as per FIGS. 1 and 2, the end region 5 of the inner tube 3 with an enlarged diameter is extended in the proximal direction by the region 5′ which covers the holes 4 in this circumferential portion.

Thus, in the design of FIGS. 1 and 2, the extended region 5′ provides a cover in a circumferential portion, while the holes 4 remain open in the remaining circumferential region. In the illustrated example, the viewing direction 9 of the optical system 8 is tilted downward from the axial direction of the outer tube 2 and the lower holes 4 of the circumferential portion situated in this tilt direction are covered.

As illustrated in FIG. 1, this results in an asymmetric liquid flow in the direction of the arrow 12. The liquid emanates with a slight downward direction in the direction of the viewing direction 9 of the optical system 8 and then runs upward to the holes 4 which are only free there, while the holes in the lower circumferential region are covered.

If the insert consisting of inner tube 3, optical system 8 and instrument 10, 11 is rotated with respect to the outer tube 2, the covering circumferential region 5′ co-rotates and so, in any angular position, the flow conditions are always formed as illustrated in FIG. 1.

FIG. 3 shows an embodiment variant of the inner tube. The same reference signs are kept if possible.

In this case, only the extended region 5′ has a different design because of the selection of a different inner tube design in this case, which is illustrated schematically.

1. A resectoscope with a shaft which has an outer tube and an inner tube, wherein an optical system with an oblique field of view is arranged within the inner tube, which system, together with the inner tube, is rotatably arranged with respect to the outer tube, wherein the distal end region of the inner tube is designed such that it can slide on the outer tube in a sealed fashion and the outer tube has holes in the distal end region proximal to the end region of the inner tube, and wherein the inner space of the inner tube is connected as a supply duct and the intermediate space between the tubes, is connected as a discharge duct, wherein the holes are arranged distributed over the circumference and the inner tube has a covering device which covers the holes in a circumferential region.

2. The resectoscope as claimed in claim 1, wherein the covering device is designed as a region which extends over the region of the holes and is part of the end region of the inner tube with an enlarged diameter.
